(19) United States
(12) Patent Application Publication
(10) Pub. No.: US 2010/0130406 A1
(43) Pub. Date: May 27, 2010

(54) GLYCATED MILK AND USES THEREOF

(75) Inventor: Robert Bartlett Elliott, Auckland (NZ)

Correspondence Address: GREER, BURNS & CRAIN, 300 S WACKER DR, 25TH FLOOR, CHICAGO, IL, 60606, US

(73) Assignee: SOMNACEUTICS LIMITED, AUCKLAND (NZ)

(21) Appl. No.: 12/597,421

(22) PCT Filed: Apr. 24, 2008
(86) PCT No.: PCT/NZ08/00092

§ 371 Date: Oct. 23, 2009

(30) Foreign Application Priority Data
Apr. 24, 2007 (NZ) 554747

(51) Int. Cl. A61K 38/10; C07K 9/00

(52) U.S. Cl. 514/8; 530/322

(57) ABSTRACT

Milk-based products and drinks are provided including specified forms of identified proteins or fragments thereof, as are methods for the use of such milk-based products and drinks in the promotion of sleep.

### FIELD

This invention relates to milk-based products and drinks including specified forms of identified proteins or fragments thereof, and to the use of such milk-based products and drinks in the promotion of sleep.

Definitions

“Soporific” is used herein as a term for a material having a sleep-inducing effect. Established terms such as “sedative” and “hypnotic” are considered to imply a relatively stronger type of material, such as a drug.

“AGE” is an abbreviation for Advanced Glycation End-product. For example the first step in glycation of a peptide or protein is the formation of a Schiff base when the aldehyde group of a glucose (or like) molecule combines with the amino group of a lysine molecule in a peptide chain forming an imine or Schiff base which has a double bond between the carbon atom of the glucose and the nitrogen atom of the lysine. The “Amadori product” is the second stage in this version of formation of an AGE, made by rearrangement, wherein the hydrogen atom from the hydroxyl group adjacent the carbon-nitrogen double bond moves to bond to the nitrogen, leaving a ketone. The last step (an irreversible step) is oxidation of the Amadori product.

Glycation is defined more generally as the result of a sugar molecule such as glucose or fructose bonding to a protein or lipid molecule without the controlling action of an enzyme.

This specification refers to exogenous glycation, occurring outside the body. Glycation of proteins tends to make them resistant to enzymatic cleaving and significantly increases their half-life in the body.

“Maillard reaction” or sequence refers to a chemical reaction between an amino acid and a reducing sugar; usually involving heat, and resulting in some extent of non-enzymatic browning as well as the development of flavours, being some of the range of “Maillard products” which are a type of AGE.

“UHT” is an abbreviation for Ultra-High Temperature treatment—a version of pasteurisation typically run at about 140 deg C. for several seconds only, known to prolong the keeping qualities of a sealed container of milk over those obtained by conventional pasteurisation.

Problem to be Solved

People sometimes or in some cases often require a soporific, but many are cautious about taking any of the relatively strong sleeping pills, especially on a regular basis when a form of dependence may arise, or of taking alcohol for which the side effects may undo any immediate effect. Nevertheless, 551,426 sleeping pill prescriptions were dispensed in New Zealand in 2005, among just under 4 million people. There appears to be a need for a “natural” sleeping draught or at least an alternative.

### BACKGROUND

That warm milk drunk in amounts of about 100-250 ml can induce drowsiness or sleep has been known for some centuries. Viewing the hits obtained in a “Google” search in April 2008 for (sleep “warm milk”) reveals many sites that simply deny the effect. More sites accept the “old wives' tale”. Some try to explain it in a variety of ways, including psychology, effects of tryptophan, and so on. However the physiological or pharmacological mechanisms, if any, that underlie this effect have not been directly established. What (if anything) might be the active ingredient in milk?

For humans, 1-tryptophane has been suggested as a soporific ingredient, but the quantity required (about 1 g) for such an effect would require drinking about 2.5 litres of milk.

Melatonin could have a soporific effect for some people in a quantity of 3-5 mg, but but the quantity required for such an effect would require drinking about 25 litres.

Calcium could be a neurosedative, but the effect is not seen with tablets.

There is no evidence for lactose having a soporific effect.

A theoretical basis for this invention is as follows. Note that this theory is offered only as guidance and the invention may be found to be effective in practice even though a mechanism or active principles unlike the ones to be described is later shown to be correct.

Experiments reported by Guesdon et al in Peptides (2006) vol 27, pp 1476-82 show a soporific effect in rats fed a tryptic hydrolysate of bovine alpha-S1 casein.

The inventor's theory assumes (at least in the first instance) that glycated versions of caseins of the types found in heat-treated A1 bovine milk are or become effective soporific substances when consumed in reasonable amounts such as about 100-250 ml of warmed milk, then are hydrolysed into peptides by gut enzymes, and then are absorbed into the body of the consumer as glycated peptides. In particular, the glycated peptide beta-casomorphin-7 is at present believed to be mainly responsible for the soporific activity, although other glycated peptides or other substances may be either directly or indirectly involved. Further, the theory proposes that glycation enhances the soporific effect over that of a non-glycated peptide, presumably by delaying removal of the, active peptide(s) from the body, while glycation does not directly mask the soporific effect. Clearly, one test for this theory is to compare the efficacy of A1 type milk against A2 type milk (which does not become converted into beta-casomorphin-7 during digestion in the human gut) which has been similarly treated. That test may show that other glycated peptides are involved.

Evidence that glycation renders peptides (in general) resistant to peptidases is available, as in the article “Enzymatic digestion and mass spectrometry in the study of advanced glycation end products/peptides” in J Am Soc Mass Spectrom. 2004 Apr.; 15(4):496-509, Lapolla A, Fedele D, Reitano R, Arico N C, Seraglia R, Traldi P, Marotta E, and Tonani R. of Dipartimento di Scienze Mediche e Chirurgiche, Cattedra di Malattie del Metabolismo, Universita degli Studi di Padova, Padova, Italy. This article reports an extensive study carried out on human serum albumen (HSA) and non-enzymically glycated HSA by enzymatic digestion with trypsin and endoproteinase Lys-C, with the aim of identifying specific glycated peptides deriving from enzymatic digestion of glycated HSA. Those peptides may be considered, as a first approximation as advanced glycation end products/peptides. These compounds, important at a systemic level in diabetic and nephropathic subjects, are produced by enzymatic digestion of in vivo glycated proteins. They are related to the pathological state of patients and have been invoked as responsible for tissue modifications. The digested mixtures obtained by the two enzymes were analyzed by the proteomics techniques MALDI/MS (matrix assisted laser desorbtion/ionisation/time of flight) mass spectroscopy) and LC/ESI/MSn (liquid chromatography electrospray ionisation tandem mass spectroscopy), and clear-cut differences were found. First of all, the digestion products of glycated HSA are generally less abundant than those observed in the case of unglycated HSA, accounting for the lower proclivity of the former to enzymatic digestion. MS/MS experiments on doubly charged ions, comparisons with a protein database, and molecular modelling to identify the lysine NH₂ groups most exposed to glycation, identified some glycated peptides in digestion mixtures obtained from both types of enzymatic digestion. Residues 233K, 276K, 378K, 545K, and 525K seem to be privileged glycation sites, in agreement with the fractional solvent accessible surface values calculated by molecular modelling.

Related articles may be obtained through the Medline (PUBMEJ)) links from the above paper (PMID: 15047055) as citations; such as “Advanced glycation end products/peptides: an in vivo investigation.” [Ann N Y Acad Sci. 2005] PMID:16037247 (same authors), and “The role of mass spectrometry in the study of non-enzymatic protein glycation in diabetes: An update” [Mass Spectrom Rev. 2006] PMID:16625652

The inventor is not aware of previous publications in which glycation of a peptide, particularly a morphin, is used in order to enhance its effect on a receptor or to delay its breakdown. On the other hand, Longobardo L, et al; Bioorg Med Chem Lett. 2000 Jun. 5;10(11):1185-8 tested two analogues of bovine beta-casomorphin-7 and beta-casomorphin-5 containing a beta-homo phenylalanine in substitution of the phenylalanine in position 3 for their mu-opioid receptor affinity. The modification enhanced the mu receptor affinity 5-fold in the case of modified beta-CM-7 and 2-fold for modified beta-CM-5 when compared to the natural peptides. Kreil G et al, Life Sci. 1983;33 Suppl 1:137-40 believe that the endogenous enzyme involved in beta casomorphin destruction is identical with or similar to dipeptidyl-peptidase IV. They found that beta-casomorphin (beta-CM) analogues in which the proline residue in position two has been replaced by D-alanine seem to be completely resistant to enzymatic attack in the plasma. Hence the altered peptide became persistent.

Object

It is an object of this invention to provide a safe and convenient soporific, or at least to provide the public with a useful choice.

Statement of Invention

In a first broad aspect this invention provides a soporific composition for consumption by humans or other mammals; the composition comprising a glycated milk of preferably bovine origin; the milk proteins of said milk having previously undergone glycation at least partly as a consequence of processing by a manufacturing process.

Preferably the product includes at least one glycated, soporific peptide or a precursor thereof; the soporific effect of which peptide when absorbed is substantially prolonged as compared to a non-glycated form of the soporific peptide or a precursor thereof. (By “substantially prolonged” the inventor means that modified (glycated) beta-casomorphin-7 derived from certain types of heat-treated and therefore relatively extensively glycated milks has a half-life of hours rather than minutes in the circulation).

In another aspect the invention provides a manufactured, edible soporific beverage or food product wherein the beverage or product includes at least one glycated protein capable, after ingestion by a person and after being hydrolysed by gut enzymes, of releasing at least one glycated, soporific peptide capable of being absorbed into the person's circulation.

Preferably the soporific composition includes substantially a glycated beta-casein A1.

Preferably the milk-based soporific composition is effective for an adult in an oral dose of 100-250 ml.

Preferably the composition is drunk after warming.

Preferably the selected glycated proteins include glycated bovine A1/A1 beta-casein.

Preferably the glycated soporific peptide is a glycated bovine beta casomorphin-7.

In a related aspect, the product is comprised of milk taken from a population comprising at least one dairy cow previously selected so as to be substantially homozygous for the A1 beta-casein gene; said milk having been processed during manufacture in order to cause at least partial glycation of proteins within the composition, so that, when digested by a mammal or human being, an effective amount of glycated soporific peptides is released and absorbed into the circulation.

Alternatively, the product is derived from milk taken from a population comprising at least one dairy cow previously selected so as to be substantially homozygous for the A1 beta-casein gene as previously described in this section; said derivative of milk including glycated bovine A1/A1 beta-casein or parts thereof and capable of releasing an effective amount of glycated soporific peptides into the circulation after digestion.

In a second broad aspect the invention provides a method for manufacturing a soporific product of the type previously described in this section wherein the method comprises (a) acquiring A1 type bovine milk, (b) adding a glycation promoting carbohydrate material, (c) causing glycation during or after a high-temperature sterilising treatment, and (d) packing the product.

Preferably the method includes further steps of (e) testing the product in order to determine the amount of glycation and (f) labelling the product according to its expected soporific effect.

Alternatively, the product after testing includes a known amount of glycated beta-casein.

In a further aspect, glycation is promoted simply by long-term storage for perhaps 1-6 months at room temperature or above of milk prepared by the “U H T” version of pasteurisation.

In a related aspect, the method includes the steps of testing the product in order to ascertain the extent of glycation at the time, and then of diluting the product to reach a consistent soporific effect as indicated by the glycation test results; the diluent comprising an equivalent product made in the same manner but using an A2/A2 milk so that each package of the product has a consistent amount of glycation and is consistent with respect to all components of the product except that the proportion of A1-beta casein in the product is varied according to the test results.

Preferably the glycation promoting material is selected from a range of materials capable of inducing glycation reactions; the carbohydrates including the range of fructose, galactose, mannose, glucose, and ascorbic acid.

In a third broad aspect the invention provides a pharmaceutical product based on glycated A1/A1 milk as previously described in this section, wherein a glycated form of the peptide beta casomorphin-7 is extracted from the glycated A1/A1 milk and packaged along with appropriate preservatives, excipients and the like as a peptide suitable for oral, intra-buccal or parenteral administration; the product providing a glycated peptide having a soporific effect.

Preferred methods of extraction of a soporific glycated peptide from a glycated beta-casein A1 involve an enzymatic hydrolysis selected from the range including: exopeptidase hydrolysis, endopeptidase hydrolysis (including peptic hydrolysis, tryptic hydrolysis, and chymotryptic hydrolysis), or a combination thereof.

In a fourth broad aspect the invention provides a clear signpost to the manufacture of specific sleep-promoting or sleep-inducing peptides that resemble peptides released from type A1 beta-casein in the mammalian (including human) digestive tract.

### Preferred Embodiment

The description of the invention to be provided herein is given purely by way of example and is not to be taken in any way as limiting the scope or extent of the invention.

Throughout this specification unless the text requires otherwise, the word “comprise” and variations such as “comprising” or “comprises” will be understood to imply the inclusion of a stated integer or step or group of integers or steps but not the exclusion of any other integer or step or group of integers or steps.

### EXAMPLE 1

This invention relates in particular to a soporific based on specific kinds of milk that includes glycation end-products convertible, preferably by endogenous gut enzymes after ingestion, into soporific glycated polypeptides. It also relates to a relatively long-acting soporific comprising a glycated peptide obtained from specific kinds of milk.

So far as is known, the only effective kind of bovine milk for the present purpose is that derived from cows homozygous for the A1 gene controlling the sequence of the milk protein beta-casein. Such cows are widespread in most dairy herds; for example the Holstein or Friesian breeds produces predominantly A1/A1 type milk whereas the Jersey breed produces predominantly either A1/A2 or A2/A2. (Other alleles of the A1 and A2 genes are known but are believed to be either unimportant or of low frequency in bovines). A mixture of A1 and A2 type milks may be effective for use according to the patent, but the soporific effect is diluted and relatively hard to predict. There are well-known DNA-based (genotypic) and phenotypic techniques for the selection of cows homozygous for the A1 gene as distinct from the A2 gene and others, and such cows will breed true when A1/A1 bulls are used over them.

Bovine milk contains about 3.4 to 4.5% proteins of which about 80% is casein. About 31% of the casein (1 g per 100 ml, approx) is beta-casein—mainly either A1 or A2 variants. After hydrolysis of A1 type beta-casein but not of type A2 beta-casein during digestion within the gut, about 1 mole of the peptide beta-casomorphin-7 is produced for each mole of casein of the A1 (or B) type, which have a histidine residue at position 67. That peptide may be capable of serving as a soporific in humans. Beta-casomorphin-7 is classed as “an opioid” and is known to have sedative and anxiolytic effects in rats, chickens and cockroaches.

Continuing to consider the list of possible soporific components as discussed in the Background section, there is sufficient of the beta-casomorphin-7 precursor(s) in a glass of milk to have a soporific effect on a human consumer. The half-life of normal beta-casomorphin-7 in the circulation is about a few minutes. It is converted into other chemical species by the body; presumably by enzymic cleavage in plasma, the liver or the kidneys.

Assuming that beta-casomorphin-7 is the active ingredient, the inventor therefore proposes that varieties of milk high in A1 casein be sold for the purpose of inducing drowsiness or sleep in “a natural way”. Milk from selected A1-A1 cows (one, or more, such as a herd or population of cows) provides about twice as much beta-casomorphin-7 as does using ordinary dairy milk as on retail sale, in which most of the remaining casein is A2-casein, with small amounts of other forms of casein.

Further, there is the matter of the short half-life of beta-casomorphin-7. The inventor has noted that modified (glycated) beta-casomorphin-7 derived from certain types of heat-treated and therefore relatively extensively glycated milks has a half-life of hours rather than minutes.

Accordingly it is easier, as will be described below, to manufacture and sell a quantity of glycated, otherwise naturally-occurring precursors of beta-casomorphin-7 as a potentiated soporific beverage based on milk than it is to synthesise a beta-casomorphin-7 having substitutions along the peptide chain for a similar purpose. This invention provides that the selected milk, having predominantly the A1 type of beta-casein) be treated so as to produce an effective amount of glycated beta-casomorphin-7. In general, treatments for milk that have an effect of enhancing the AGE content, or otherwise raising the proportion of glycated beta-casein in a product, include:
1. heating the milk for a longer period during pasteurisation or more preferably before, during and/or after the UHT version of pasteurisation, (or providing a modified pasteurisation or UHT process),
2. storing a sterile milk at a raised or at room temperature for an extended period,
3. adding ascorbic acid or fructose, galactose or glucose (among other sugars) in combination with storage, before or after sale. Fructose (also known as laevulose) and galactose are said to be about ten times as active as glycation agents as is glucose, but glucose is commonly available. The range of useful glycation-enhancing materials in milk includes ascorbic acid.
4. Components as named in (3) above may be added before the heating/pasteurisation step.
5. Testing the resulting product in order to confirm the content of glycated material therein. The FAST index is one example test method for detecting early glycation products, and an assay for carboxymethyl lysine is one appropriate test for AGE products. The product as manufactured should preferably include at least a consistent amount of soporific ingredients, although it is known that the amount tends to rise during storage and this effect should be allowed for when calculating a shelf life.

Treatments of milk or milk products resulting in glycation have occurred incidentally in the past. When tested according to the FAST index method, pasteurised whole milk (of undefined A1/A2 status) simply held in storage at 4 deg Celsius shows a rise in AGE-related compounds as follows: 24 hrs-10; 48 hrs 10.7; 72 hrs-17.4; and 1 week: 22.6. Also, the browning that is characteristic of the Maillard reaction has been noted in UHT milk that has been simply stored for a period according to recommended conditions.

Therefore the inventor proposes that in order to manufacture an effective soporific or sleeping draught, milk from A1-A1 cows that has been glycated by heating in the presence of specified carbohydrates and put through the “UHT” process be sold for the purpose of encouraging sleep, because the glycation process renders the subsequently absorbed beta-casomorphin-7 relatively resistant to conversion into inactive forms within the human body. The effect of further storage on efficacy is generally to increase the amount of glycation. The efficacy of the substance as produced commercially may be expressed in terms of the amount, as established by test, of glycated protein or glycated beta casein contained therein.

The milk may be sold in single-dose cardboard cartons similar to those for UHT milk, and it would be handy if the cartons were compatible with heating in a microwave oven or by dropping, still sealed, in hot water, so that the milk drink could be warmed before consumption. The benefits of warming may be purely psychological as by forming part of a ritual of going to sleep.

Of course it is known that glycated foodstuffs may be harmful, especially to diabetics although the total amount of glycated material is small. Therefore this invention should be used with care by the elderly or by diabetics or by persons undergoing dialysis. Preferably the soporific product is sold with a recommended dose statement and with a warning against carrying out risky actions such as driving or operating machinery for a time after consuming the product.

### EXAMPLE 2

A non-limiting example method of manufacture is provided as follows:
1. Pour bovine milk that has been obtained from cows predominantly or preferably all having the A1/A1 beta-casein phenotype into a processing tank.
2. Add between 1 and 2 grams/liter (0.1 to 0.2%) of sodium citrate, as a stabiliser.
3. Stir until the citrate is dissolved.
4. Add 1 gram/liter of ascorbic acid (0.1%) as a glycation promoter.
5. Stir and heat over about 20 minutes to reach 45 degrees Celsius
6. Hold at 45 deg Celsius for 30 minutes
7. Homogenise using standard industry homogenisation technology
8. Apply UHT treatment at 141 deg C. for 4 seconds.
9. Sterile fill packs with the UHT-processed liquid and seal them.
10. Consumers are recommended to warm the milk before drinking it.

The inventor expects much of the glycation reaction to occur during and just after the above process, particularly while the composition is hot, although glycation is capable of continuing during storage at room temperature or even during refrigeration. Quality assurance/quality control of the manufacturing process is provided in one aspect by monitoring of the pH, and Table 1 shows a set of acceptable pH results.

TABLE 1

| | | |
|---|---|---|
| Stage | Temperature | pH |

| | | | |
|---|---|---|---|
| Raw milk accepted | 16 deg | C. | 6.82 |
| After adding citrate | 15.8 deg | C. | 6.74 |
| After adding ascorbic acid | 15 deg | C. | 6.64 |
| After homogenisation | | | 6.73 |
| After UHT treatment | 17.2 deg | C. | 6.42 |

Other forms of quality control include measurement of the extent of glycatidn at the time of manufacture. Only if all the milk is obtained from cows all having the A1/A1 phenotype is it possible to anticipate the soporific effect of a manufactured product. It may be advisable to anticipate further glycation during transport to market. A comparison of the “sleep milk”, as produced by this invention, with other forms of milk is in the following table 2, which shows results from several tests well-known to workers in the art, (a) Furosine in mg/ml (a measure of early glycation products); (b) FAST index (a measure of late glycation/Amadori products, and (c) CML in ng/ml (carboxymethyl lysine test - a surrogate marker of AGE. Note that those tests do not specifically identify the precursors of beta-casomorphin-7. If pure A1/A1 milk was used, the correlation between an AGE-related test and a soporific effect is more certain. According to the inventor's theory, milk derived from A2/A2 cows that was processed as above would have little soporific effect.

TABLE 2

| Sample | Furosine mg/ml | FAST index | CML ng/ml |
|---|---|---|---|
| Raw milk | 4.4 | — | — |
| Pasteurised milk | 9.5 | 10 | 2.9 |
| Pasteurised milk with | — | 19.3 | — |
| ascorbic acid (1 g/litre) | | | |
| UHT milk | 20.5 | 17.5 | — |
| “Sleep milk” (the | 22 | 22.5 | 5.91 |
| invention) | | | |

The inventor realises that the glycation process is relatively complex, and that this account of the preparation of a “sleep milk” has not taken into account all the aspects of glycation. Nevertheless this specification sets out the principles.

Variations

1. Beverages having an equivalent functional (glycated morphin) basis, although not made of milk. Gliadomorphins may be equivalent to beta-casomorphin-7 in terms of having a soporific effect. One well-known beverage having a claimed soporific effect is the product known as “Horlicks” (TM) (made by Glaxo SmithKline) which includes, usually with milk, malted barley and wheat that has been heat-dried. Malting releases glucose which can combine with the barley and wheat proteins to produce glycated products. On digestion, these glycated proteins will release either gliadomorphins or glycated gliadomorphins. Whether such gliadomorphins have a useful soporific effect remains to be assessed.

2. In order to reduce a soporific effect of a product according to the invention down to a standard amount preferably after a test for AGE-related components, some of the milk used in making that product may be obtained from A2/A2 phenotype cows, so that the general (such as taste and nutritional) characteristics of the product remain consistent yet the soporific effectiveness may be tailored. The beta-caseins of an A2/A2 cow do not contribute the active beta-casomorphin-7.

3. The glycation promoter mentioned in the method may be selected from a range of carbohydrates capable of inducing glycation reactions; the carbohydrates including fructose, galactose, mannose, and glucose, and ascorbic acid.

4. Derivatives of a basic milk product—such as an A1 milk derivative from which the fats and/or the sugars have been removed or at least partially removed during processing, typically before undergoing a glycation process. Flavourings may be added. These enhancements are based on the observation that many modified forms of “whole milk” wth various amounts of customer appeal are now on sale.

5. Variations such as a product containing glycated beta-casein alone, or one or more glycated synthetic polypeptides alone. The Longobardo and Kreil publications mentioned above suggest that if the substituted beta casomorphin-7s described in either of those publications were also glycated, their effects would be enhanced. Such peptide may be refined from natural sources, made by genetically modified micro-organisms, or synthesised from amino acids, as is found convenient within a manufacturing environment, then are glycated, and then sold in a stable and acceptable form such as in a sterilised beverage or as a pharmaceutical product for oral, trans-buccal or parenteral administration. Such a product is at least of use to those who have an allergy to bovine milk. In the event that a 7-amino acid peptide by itself cannot easily be glycated, manufacture will be directed to a larger peptide, the sequence and configuration of which provides that it will be cleaved at positions during enzymic hydrolysis in the gut that result in the release of a beta casomorphin-7 analogue.

6. Developed from the above option, the invention also provides a glycated peptide that has a soporific effect. This powder may be manufactured as a powder or a sterile solution, and distributed for use as a pharmaceutical product rather than a modified food, The active material is provided along with suitable excipients and carriers for oral, trans-buccal or parenteral administration. In this option, the list of suitable glycated peptides includes glycated beta casomorphin-7. The pharmaceutical product may be manufactured from glycated A1/A1 milk as previously described by in-vitro protein hydrolysis (such as an enzymatic hydrolysis selected from the range of proteases including: exopeptidase hydrolysis, endopeptidase hydrolysis (including peptic hydrolysis, tryptic hydrolysis, and chymotryptic hydrolysis), or a combination thereof, followed by extraction of peptides of the appropriate mass, or it my be made by commencing with an extracted or synthesised beta casomorphin-7 or like, or precursor peptide that is then glycated.

### INDUSTRIAL APPLICABILITY AND ADVANTAGES

The invention provides a purified and enhanced form of an existing substance, since ordinary bovine milk inevitably including some glycated beta-casein Al-type milk sold in UHT processed form has been available for some time. Therefore there should be no objection to sales to the public of a “designed” product including similar components especially if the product has been tested so as to have a defined, consistent amount of soporific activity as well as a having nutritional value.

Advantages of the invention over existing sedative and hypnotic pharmaceuticals include: cost, probable absence of any dependency, and reliance on natural ingredients and a common process, while providing an outlet for value-added dairy products.

Contra-indications may include the presence in a consumer of clinical or undiagnosed diabetes or other syndromes particularly susceptible to the ingestion of AGEₛ. Use of functionally specific materials such as those outlined may make very little difference to a person's total intake of glycated protein-based materials. The dose of glycated proteins or fragments thereof is at most about 3.4 to 4.5 g per 100 ml of milk in a soporific dose, assuming total glycation.

Finally, it will be understood that the scope of this invention as described by way of example and/or illustrated herein is not limited to the specified embodiments. Where in the foregoing description, reference has been made to specific components or integers of the invention having known equivalents, then such equivalents are included as if individually set forth. Those of skill will appreciate that various modifications, additions, known equivalents, and substitutions are possible without departing from the scope and spirit of the invention as set forth in the following claims.

1. A manufactured product having a soporific effect after introduction into a mammalian body, comprising; at least one glycated, soporific peptide or a precursor thereof; the soporific effect of which glycated peptide is substantially prolonged as compared to a non-glycated form of the soporific peptide or a precursor thereof.

2. The product as claimed in claim 1, characterized in that the glycated peptide is a glycated bovine beta casomorphin-7.

3. The product as claimed in claim 1, characterized in that the beverage or product includes at least one glycated protein capable, after ingestion by a mammal and after being hydrolysed by gut enzymes, of releasing at least one glycated, soporific peptide capable of being absorbed into the mammal's circulation.

4. The product as claimed in claim 3, characterized in that the selected glycated proteins include glycated bovine A1/A1 beta-casein.

5. The product as claimed in claim 4, characterized in that the product is comprised of milk taken from a population comprising at least one dairy cow previously selected so as to be substantially homozygous for the A1 beta-casein gene; said milk having been processed during manufacture in order to cause at least partial glycation of proteins within the composition, so that, when digested by a mammal, an effective amount of glycated soporific peptides is released and absorbed into the circulation.

6. The product according to claim 4, characterized in that the product is derived from milk taken from a population comprising at least one dairy cow previously selected so as to be substantially homozygous for the A1 beta-casein gene; said derivative of milk including glycated bovine A1/A1 beta-casein or parts thereof; said derivative of milk having been processed during manufacture in order to cause at least partial glycation of proteins therein so that, when digested by a mammal, an effective amount of glycated soporific peptides is released into the circulation.

7. The manufactured soporific beverage or product as claimed in claim 5, characterized in that the product includes a known amount of glycated beta-casein.

8. A method for manufacturing a soporific product of the type claimed in claim 5, comprising: (a) acquiring A1 type bovine milk, (b) adding a glycation promoting carbohydrate material or ascorbic acid, (c) causing glycation during or after a high-temperature sterilizing treatment, and (d) packing the product.

9. The method for manufacturing a soporific product as claimed in claim 8, further including the steps of (e) testing the product in order to determine the amount of glycation and (f) labeling the product according to its expected soporific effect.

10. The method for manufacturing a soporific product as claimed in claim 8, further including the steps of (e) testing the product in order to ascertain the extent of glycation, and then of diluting the product to reach a consistent soporific effect as indicated by the glycation test results with an equivalent product made in the same manner but using an A2/A2 milk so that each package of the product has a consistent amount of glycation and is consistent with respect to all components of the product except that the proportion of A1 betacasein is varied.

11. The method for manufacturing soporific product as claimed in claim 8, characterized in that the glycation-promoting material is selected from a range of materials capable of inducing glycation reactions; the carbohydrates including the range of fructose, galactose, mannose, glucose, and ascorbic acid.

12. The use of glycated bovine A1/A1 beta-casein for the preparation of a medicament for the treatment of insomnia.

13. A pharmaceutical product based on glycated A1/A1 milk as claimed in claim 1; characterized in that a glycated form of the peptide beta casomorphin-7 is extracted from the glycated A1/A1 milk and packaged as a peptide suitable for oral, intra-buccal or parenteral administration; the product providing a glycated peptide having a soporific effect.
